(19) United States
(12) Patent Application Publication
(10) Pub. No.: US 2010/0330612 A1
(43) Pub. Date: Dec. 30, 2010

(54) BIOCHIP FOR ELECTROPHYSIOLOGICAL MEASUREMENTS

(75) Inventor: Claus Birger Sorensen, Dyssegaard (DK)

Correspondence Address: GIFFORD, KRASS, SPRINKLE,ANDERSON & CITKOWSKI, P.C, PO BOX 7021, TROY, MI, 48007-7021, US

(73) Assignee: Inxell Bionics ApS, Kobenhavn (DK)

(21) Appl. No.: 12/918,503

(22) PCT Filed: Feb. 21, 2008
(86) PCT No.: PCT/IB2008/050639

§ 371 Date: Aug. 20, 2010

(51) Int. Cl. C12Q 1/02; C12M 1/34; B05D 5/12

(52) U.S. Cl. 435/29; 435/287.1; 427/58

(57) ABSTRACT

The present invention relates to a substrate for measuring the electrophysiological properties of ion-channels located in cell membranes. The substrate is typically used in a screening device providing high-throughput industrialized measurements for studying ionic currents, particularly useful in the screening of drugs acting on the ion-channels found in cell membranes, by providing many parallel simultaneous and independent measurements. The substrate has one or a plurality of individually addressable electrode sites, each comprising one or more individual elongated nanosize electrodes, capable of penetrating the cell membranes during application of cells directly on the substrate, thus providing one or more low resistance contacts to the interior of the cells. This allows for an easy and effective low-cost solution to automated patch clamp measurements in the whole-cell configuration, using both single as well as multi-electrode contacts to each cell. The invention also makes ensemble measurements on multiple cells in parallel possible.

### FIELD OF THE INVENTION

The present invention relates to a substrate and method for measuring cellular electrical activity, both spontaneous and stimulated. More specifically, the invention relates to a substrate for measuring the properties of ionic currents through lipid membranes of living cells and/or potentials across such membranes, by establishing a configuration suitable for performing electrophysiological measurements.

More particularly, the present invention relates to a substrate that is typically used in a screening device providing high-throughput industrialized measurements for studying ionic currents, particularly useful in the screening of drugs acting on the ion-channels found in cell membranes, by providing many parallel simultaneous and independent measurements.

### BACKGROUND OF THE INVENTION

Ion channels are proteins found in lipid cellular membranes, where they control the flow of ions through the cell membranes. The flow through the channel is controlled by a “gate” which can be activated or deactivated by e.g. chemical or electrical signals. These ion channels play an important role for a number of processes mainly in the nervous system such as synaptic signalling, motion of muscles, regulation of hormones etc. A number of diseases are related to the dysfunction of these ion channels, which makes these channels an important target for drugs which act as modulators of these channels. A few well known examples of such diseases are different forms of epilepsy, migraine, osteopetrosis, cardiac arrhythmias, cystic fibrosis and kidney stones. The number of known diseases related to the dysfunction of ion channels has grown drastically, (C. A. Hübner et al., Human Molecular Genetics 11, p 2435-2445, 2002) since the existence of ion channels was confirmed in the 1970s by Neher, Sakmann and Steinback (Pflüger Arch. 375, p 219-278, 1978). Neher and Sakmann were awarded the 1991 Noble Prize in Physiology and Medicine for their work in developing a method for measuring ion channel transport by an electrical recording technique: The patch clamp technique.

The patch clamp technique is now one of the most popular methods for measuring the ion transport across cell membranes. It provides a method for direct measurement of the electrical signals related to the ion transport in a single cell. The first experiments were done, as shown in FIG. 1, by pressing a thin glass pipette 1 (diameter less than about 2 μm), filled with at suitable saline solution, against the cell membrane 20. By measuring the current between an electrode 2 located inside the pipette 1 and an electrode 3 located in a bath in which the cell is contained, small changes in the electrical current was observed. The discrete jumps in the electrical current that was observed were attributed to the opening or closing of ion channels. One severe limitation of these first experiments was the poor seal resistance obtained between the pipette and the cell membrane. The resistance of these first seals were in the order of ten's of MΩ, a very small resistance compared to the resistance of a single ion channel which is typically around 10 GΩ. The poor seal resistance obscured high quality measurements of the low-level currents in the ion channels. The large and often varying leakage current flowing in the poor seal also prevented a proper voltage clamping of the membrane potential for the following reason: The true membrane potential is the voltage across the resistor Rₘ 30 as shown in FIG. 2. This voltage is the result of a voltage division of V_{CMD} with the ratio between Rₘ and Rₛ+Rₘ. In the case of a leak, one may model the leak by a resistor connected in parallel to Rₘ. The result will be a change of the true membrane potential.

Improvements of the pipette based patch clamp technique eventually lead to the realization of high resistance seals of more than 1 GΩ (so called Gigaseals). These high quality seals reduced the current noise dramatically and facilitated clamping of the membrane potential to a value desired by the experimentalist. The refinement of the pioneering patch clamp work was done by: O. P. Hamill, A. Marty, E. Neher, B. Sakmann and F. J. Sigworth and was published in: Pflügers Arch. 391, p 85-100, 1981.

Standard laboratory patch clamp techniques still rely on the use of a single glass patch pipette which is positioned by a micro-manipulator on a single cell membrane by a person skilled in the art of electrophysiology. This makes the testing of drug candidates acting on ion channels a very time consuming and expensive process, effectively preventing the testing of a large number of potential drug candidates.

In a typical drug discovery process a vast amount of compounds are initially fabricated, but only a few are selected for further screening, due to the natural limit of resources available.

The discovery of a single drug requires a vast amount of decisions to be taken, based on very limited data, which may disqualify potential drug candidates, as resources are not available for running thorough testing of thousands or more compounds. One may therefore easily overlook potentially successful candidates among the many compounds being disqualified at an early stage in the process. Another time consuming process is the safety screening of drugs, here a more industrialized approach would shorten the screening process, making the “time to market” much shorter.

An automated method for performing high quality patch clamp measurements is thus highly attractive for the pharmaceutical industry as well as for smaller volume testing, typically done in a university laboratory. An attempt at automating the process e.g. by the use of computer guided pipette positioning using machine vision was disclosed in WO 98/50791 by the company Neurosearch. This method has however proven difficult and not very successful for high volume screening.

In the last few years so called planar chip based patch clamp methods, suitable for high throughput screening have been developed. In these systems the pipette is replaced by a planar substrate of e.g. silicon as disclosed by the company Sophion Bioscience (U.S. Pat. No. 6,682,649 and U.S. Pat. No. 6,932,893), glass (N. Fertig et al. in Phys. Rev. E. 64, p 040901, 2001) or a silicone polymer: polydimethylsiloxane PDMS (U.S. Pat. No. 6,699,697). These systems typically consist of a substrate having one or more measurement sites where cells are positioned and electrophysiological measurements are performed. The sites are generally made as small holes (orifices) through a substrate, with an orifice diameter in the range of 0.5 to 3 um, onto which the cell is positioned by means of e.g. suction, a schematic example is shown in FIG. 2. The substrates 10 having a first side onto which the cells 20 are applied, analogous to the bath containing the extra cellular saline solution in traditional patch clamp, and a second side corresponding to the inside of the patch pipette containing the intra cellular saline solution. The surface around the orifice 15 may have a special coating or it may simply be a clean oxide surface of e.g. SiO₂ suitable for cell adhesion and sealing. The electrical resistance 32 may be measured by the use of a set of electrodes positioned on each side of the substrate. The resistance measured across an open orifice corresponds roughly to the resistance of the electrophysiological buffer solution and is typically in the low MΩrange. This resistance corresponds to the resistance of an open patch pipette used in traditional patch clamp technology.

When a cell 20 is positioned on a measurement site the membrane forms a seal with a high electrical resistance. If the resistance rises above 1 GΩ the term Gigaseal is used. The formation of a Gigaseal is important for high quality measurements as mentioned above. Having formed the Gigaseal and assuming that the cell membrane is still intact, the cell is in the so called “on-cell” configuration. In this configuration there could be a few ion channels located in the small membrane patch attached across the orifice. These few ion channels may be used for measurements of single ion channel events, but this on-cell configuration is mostly of academic interest. For screening of drugs the preferred measurement configuration is the “whole cell” configuration. In the “whole cell” mode a low resistance electrical access to the inside of the cell is required. This is achieved e.g. by applying short suction or electrical pulses until the cell membrane is ruptured or by adding a pore forming compound to the intracellular buffer solution on the second side. The latter method is slow and usually yields a much higher access resistance to the cell and the application of electrical pulses is not reliable, thus the suction method is often preferred. Rupturing the membrane gives electrical access to all of the ion channels located in the entire lipid membrane. The electrical access is made via contact between the intracellular solution and the electrophysiological saline solution on the second side, making up the contact between the intra-cellular metal electrode (typically made of Ag/AgCl) and the cell. Although one aims at having a saline solution on the second side of the planar patch clamp chip with an ionic composition as close as possible to the native intracellular composition, it is never exactly the same. After the cell membrane is broken, the two solutions intermix causing a change in the balance of the cells intracellular solution. This change affects the function of ion channels and the lifetime of the cell, as cellular organs and cytoplasm is lost from the cell.

WO 2004/041996 discloses a substrate consisting of intracellular electrodes located in the centre of the orifices. In one embodiment described, the above mentioned problem with intermixing is prevented by the use of an electrode with a nanometre size diameter and hydrophobic properties; so the membrane of a cell will be penetrated by the sharp electrode and a tight seal formed around the electrode after the cell is positioned on the orifice by the use of suction. The realization of such a system and the placement and fabrication of the nanosize intracellular electrode into a complex flow-channel system is however not straightforward and has to our knowledge not been demonstrated.

US 2005/0266478 also disclose the use of “needle electrodes” made of conductive material for the penetration of cell membranes. Also in this case the cells need to be applied and positioned accurately over the electrode using complex methods i.e. dielectrophoresis, requiring many electrical electrodes and contacts on the substrate. Another challenge in the present technologies is the total access resistance to the cell, also called the series resistance (Rₛ) which is typically in the range of 5-20 MΩ. Out of these up to 20 MΩ, 1-3 MΩrelates to the patch orifice and the rest to the increase in resistance related to parts of the cell membrane etc. which clogs up the patch hole during the experiment.

As shown in the zoom of the orifice in FIG. 2, the resistance of the saline solution in the narrow channel going through the membrane on the second side, and leading to the patch orifice, dominates the resistance of an open orifice and is given by:

$${R}_{access} = ρ \times \frac{l}{A} ,$$

where ρ is the resistivity of the buffer solution, l is the length and A the cross sectional area of the orifice having a diameter d. The resistivity of the saline buffer solution is typically around 71Ω×cm. A thickness of the membrane for the patch orifice of 10 μm and an orifice diameter of 2 μm, thus gives a resistance of about 2 MΩ.

Changes in the series resistance during the experiment causes problems as the voltage drop developed across Rₛ results in a change in the applied membrane potential. A whole cell current of e.g. 30 nA will develop a voltage drop of 60 mV across a 2 MΩ series resistance. This voltage drop should be compared to commonly applied membrane potentials of ±100 mV. The lack of proper voltage clamping may result in total loss of voltage control, resulting in cell death. Even if the cell does not die, series resistance errors of more than a few mV may cause very serious erroneous conclusions in the interpretation of the final results. The error could affect the voltage dependent ion channels, causing more or less of them to open or close, which makes the activation look either slower or faster as a function of the applied potential.

Another problem related to the series resistance is the time constant of the measurement system. With reference to FIG. 2 one may note that the time constant for charging the membrane is the product of the membrane capacitance Cₘ 31 and Rₛ32. High Rₛ will give a long time constant for charging the membrane to the desired potential, thus making the observation of fast ion channel dynamics difficult.

In addition to the above mentioned problems, measurements are often made difficult by the parasitic capacitance Cₚ 35 and 36. In a traditional patch clamp setup the parasitic capacitance is the capacitance between the saline solution inside the pipette and that of the bath. In planar chip based setups the parasitic capacitance is the capacitance between the saline solution on the first side (extra-cellular) and that of the second side (intra-cellular). In traditional pipette based patch clamp this capacitance is usually small (<5 pF) and it can be made even smaller by coating the pipette with e.g. Sylgard. The parasitic capacitance can be compensated for in the electronic setup, but even small changes during the measurements results in severe distortions of the measured current response from the cell.

In this relation silicon based planar patch chips present a particular challenge as insulating silicon substrates are not available. As shown in FIG. 2 the parasitic capacitance in silicon based chip is actually a combination of two capacitances 35 and 36 connected in series via the conducting substrate. The dielectric oxide layer 14 on the first and second side of the chip is usually thin (around 1 μm), and the area of the chip exposed to the saline solutions is often large, several mm². In the simple parallel plate capacitor model, one should consider the saline solutions on both sides of the substrate as being the conducting plates. The resulting parasitic capacitance of silicon chips is therefore large compared to traditional pipettes or planar glass chips.

The capacitance is given by:

$${C}_{p} = {ɛ}_{r} {ɛ}_{0} \frac{A}{t} ,$$

where ∈ᵣ is the relative dielectric constant, ∈₀ is the dielectric constant of vacuum (8.85×10⁻¹² F/m), A the area of the dielectric exposed to the saline solution and t the thickness of the dielectric.

Assuming a relative dielectric constant for SiO₂ of 4.5 and an oxide thickness of 1 μm yields a parasitic chip capacitance of about 40 pF/mm² for each of the two layers. The series connection reduces this to about half, but the exposed area of saline is in the order of a few mm², giving large parasitic capacitances of 20-100 pF. An example of the artefacts arising from the parasitic capacitance found in silicon based chips can be seen in FIG. 28B disclosed in US 2005/0266478. Here one clearly sees the narrow capacitive spikes in the current traces, arising when the applied voltage changes rapidly.

The cells used for planar patch clamp testing are usually required to be in a suspension, from which the cells are applied to the first side of the substrate by a pipette.

Prior to use, the cells are cultivated on a separate substrate in e.g. a Petri dish under suitable conditions in an incubator. To get the cells into suspension therefore requires a process step to remove them from the substrate on which they were cultivated (harvesting). This step could e.g. be an enzyme treatment that “cuts” the proteins bonding the cells to the substrate (trypsination). The use of trypsin and the resuspendsion of the cells may affect the properties of ion-channels (see e.g. K. Lee et al. Mol. Pharmacol. 46, p 176, 1994) resulting in potential sources of error. Avoiding the trypsination step could therefore be an advantage.

### SUMMARY OF THE INVENTION

The present invention provides a substrate and a method suitable for direct measurement of ion channel currents through lipid membranes of e.g. living cells or other ion channel containing structures, such as artificial membranes, especially suited for, but not limited to, the use in high throughput drug screening instruments. The invention addresses several of the above mentioned problems found in planar chips, based on substrates with orifices as well as standard patch clamp systems based on glass pipettes.

The invention provides a substrate having one or more individual sites with suitable electrode structures for determination and/or monitoring of electrophysiological properties of ion channels. The individual sites comprises one or more nanometre size elongated electrode structures (working electrodes) protruding from the substrate. The working electrodes are of such shape and size, that when a cell is applied to the substrate and adheres to the surface of the electrode structures or the surrounding surface, the cell membrane is penetrated by the thin needle-like electrode structures protruding from the substrate surface, thus providing a low resistance electrical contact to the inside of the cell. This penetration of the cell membrane occurs as the cell uptakes the electrodes without any external force applied to the cell or electrodes.

The substrate thus provides simple and low cost means for contacting the cells directly by either culturing the cells on, or applying them to, the substrate comprising the nanosized elongated electrode structures, without the need of complex and expensive electrical or mechanical positioning means and hence also without complex flow-channel/pore and orifice designs.

The leakage of intracellular solution from the cell no longer occurs as the cells form a tight seal around the electrode structures and the electrodes provide a much lower access resistance to the cell, than the ordinary “saline bridges” used both in planar and pipette based patch clamp technologies. Preventing the leakage of intracellular solution is also expected to extent the “lifetime” of the cells during testing.

The present invention could thus eliminate the need for providing the cells in suspension to the biochip, thus eliminating the need for trypsination. The cells could however also be applied to the biochip after trypsination. Moreover the individual sites provides easy means of performing either single cell measurements or so called ensemble measurements on several cells in parallel. The substrate and method of the invention constitutes a complete biochip for electrophysiological measurements without the need for a separate intracellular saline solution and flow channel system suitable for contacting the inner part of a cell or artificial membrane. The small size of the elongated nanosize electrodes also makes multiple independent contacts to the same cell possible, thus completely eliminating the effect of the series resistance, through the possibility of making three or four terminal measurements.

Furthermore the present invention solves the problems associated with the parasitic chip capacitance found in planar chip based patch clamp setups.

Hence, the invention described above provides a biochip comprising:
a. a substrate having a first side and second side, where the surface of the first side is suitable for cultivation and adhesion of living cells in a suitable carrier liquid.
b. one or a plurality of individual sites on the first side, each comprising a single or a group of elongated nanosize electrode structures suitable for the penetration of, or uptake by, the biological membranes, each electrode site and adjacent surface being capable of forming a high resistance seal with a biological membrane, thereby isolating the intra-cellular electrode structures penetrating the cell membrane from a reference electrode in contact with the extra-cellular saline solution.
c. one or a plurality of electrical connection leads connecting the elongated nanosize electrode structures to an appropriate electronic measurement setup, with the intend to monitor or apply electrical potentials or currents across the ion channel containing lipid membrane, using standard processing techniques known in the art of thin film and semiconductor processing, such as photolithography, etching and suitable deposition techniques.
d. one or more reference electrodes located either on the first side of or outside the biochip, but in both cases in electrical contact with the carrier liquid.

The substrate may be of any insulating, semiconducting or conducting material or a combination thereof such as, plastic, ceramic, or oxide or any elemental or compound semiconductor such as, Si, Ge, GaAs, GaP, InAs, InSb, SiC, or GaN.

In the case of a semiconducting or conducting substrate, adequate electrical isolation between the reference electrode and working electrode must be provided by means commonly know to persons trained in the art.

The working electrodes, realised as elongated nanosize electrodes may be made of one or a combination of conducting or semiconducting materials such as, carbon, elemental metals like Ag, Au, Pt, or Ni, or metallic alloys or metallic halides such as, AgCl, or any elemental or compound semiconductor such as, Si, Ge, GaAs, GaP, InAs, InSb, SiC, or GaN.

The conductivity of the working electrodes may be modified by the use of dopants. In general it is advantageous to have electrodes with a high conductivity. The electrodes may therefore be doped n- or p-type or a combination thereof or left undoped, whichever will provide the desired level of conductivity.

The working electrodes may be amorphous, crystalline, substantially mono-crystalline or poly-crystalline.

The height of the working electrodes may be in a range larger than a typical lipid membrane thickness, but smaller than a typical cell diameter for the cell type intended to be investigated. Thus the length may typically be in the range such as 10 nm to 10 μm.

The diameter of the working electrodes should be in the nanometre range. More specifically this diameter may be in the range between 0.8 nm to 1 μm. The diameter should be optimised to enhance the process whereby the cells under investigation absorb the electrodes from the outside and thus uptake the electrode structures through the membrane by some form of endocytosis. Different diameters may be used for different cell lines, or as a mean to selectively contact cells of a certain type, size or shape.

The working electrodes may have a coating of a compound provided to facilitate the uptake by the cell.

The penetration of the cell membrane after cultivation of cells on the substrate with the working electrodes could also be made by applying short electrical pulses between the working and reference electrodes. The electric field will be highly concentrated at the tip of the working electrode, therefore a series of short voltage pulses, applied between the working electrode and reference electrode, while monitoring the current flow or resting potential and interrupting the pulses whenever the said current or potential exceeds a predetermined threshold value, thereby rupturing a part of the cell membrane in close proximity to the tip. This method thus provides possible means for obtaining an intracellular contact to cells that will not uptake the working electrodes naturally, prior to the measurements.

The reference electrodes may be made of mixed compounds of metals and metallic halides such as, Ag/AgCl or other suitable materials known to persons trained in the art of electrophysiology.

A single biochip may comprise one or a plurality of sites having a set of working electrodes and an appropriate number of connectors to facilitate the interfacing to an external measurement setup. For single electrode measurements on single cells the spacing between individual working electrode sites should be larger than a typical cell diameter. For multi-electrode measurements such as e.g. three point measurements each cell is intended to be contacted internally by two sites of working electrodes while one reference electrode is in contact with the carrier liquid surrounding the cell. In this case the distance between the two sites comprising the working electrodes should be smaller than a typical cell diameter, while the distance between individual pairs of multi-electrode sites should be larger than a typical cell diameter.

### BRIEF DESCRIPTION OF THE DRAWINGS

FIG. 1 is a schematic representation of a pipette based patch clamp setup as described by prior art, showing a cell 20 located in an extra-cellular buffer solution 4. The cell is contacted by a pipette 1 containing an electrode 2 connected to a current to voltage converter 8. The command voltage V_{CMD} is applied to the cell via resistor 5 and electrode 3.

FIG. 2 is a schematic representation of a planar patch chip based substrate as described by prior art. Also shown in the figure is the equivalent electrical circuit, constituting the cell and the planar patch chip and a zoom of the patch orifice.

FIG. 3 to FIG. 7 are examples of schematic representations of different devices and embodiments of the present invention.

FIG. 8A-F schematically shows procedural steps for fabrication of a single region containing the elongated nanosize electrodes.

FIG. 9 is a scanning electron micrograph of a sample with nanorods grown by Molecular beam Epitaxy.

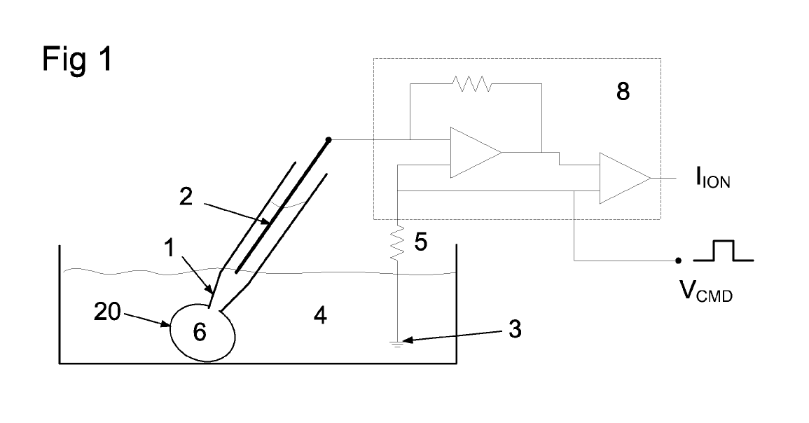

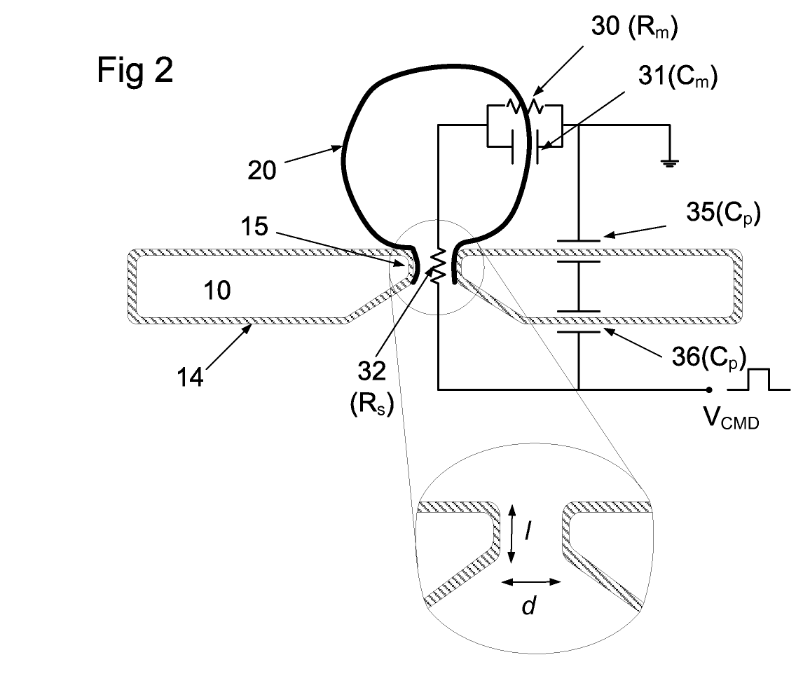

### DESCRIPTION OF THE DRAWINGS/EMBODIMENTS

The substrate according to the present invention is preferably designed for the parallel processing of a large number of electrical tests on ion channel containing membranes or cells simultaneously. This is accomplished by providing a substrate having a plurality of individual sites, each designed for contacting a single membrane or cell. Each site, constituting a set of working electrodes, where the intra-cellular electrode is a single or a set of elongated nanosize electrodes and the extra-cellular electrode is a standard electrode as commonly used in the art, such as Ag/AgCl. Each working electrode set is connected via appropriately designed integrated conductors to facilitate the interfacing to an external measurement setup. This external setup also comprises means for supplying test compounds, buffer solutions and appropriate electronics for performing the electrical measurements.

According to the present invention the substrate can have a number of different configurations. In the following descriptions any ion channel containing lipid membrane on which one may measure electrophysiological properties is for simplicity denoted “cell”. The figures are schematic examples of some preferred embodiments and should not be considered as the only way to realise a working device, any person skilled in the art of semiconductor processing would be able to find alternative techniques for making the appropriately needed lead-in connections etc. to the elongated nanosize electrodes. The following descriptions are to be interpreted in the light of the accompanying claim set. In the context of the claims, any terms such as “comprising” or “comprises” are not intended to exclude other possible elements or procedural steps. Likewise the use of “a” or “an” should not be considered as excluding a plurality. Furthermore, individual features mentioned in different claims, may possibly be combined, and the mentioning of these features in different claims does not exclude that a combination of features is not possible and advantageous.

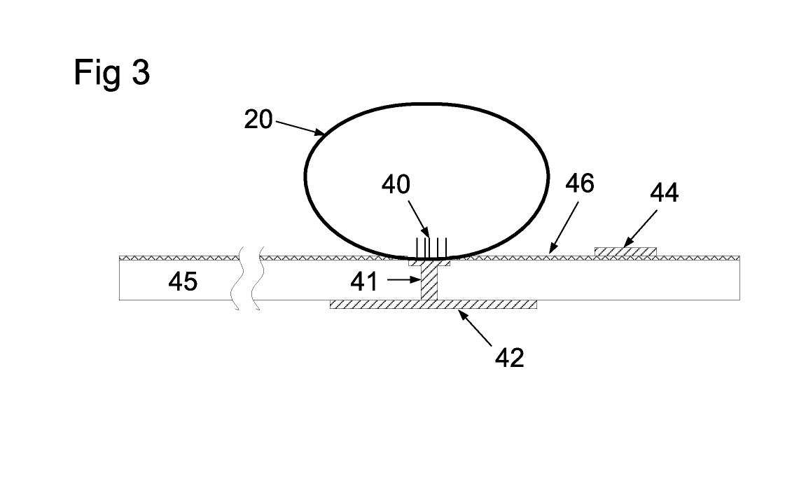

FIG. 3 shows a substrate 45 with a single cell 20 attached to the surface 46. The cell forms a tight seal on the surface and/or around the elongated nanosize electrodes 40 which penetrates the membrane, thereby making contact to the intra-cellular solution of the cell. The intracellular electrodes 40 are electrically connected to a contact 42 on the second side of the substrate by means of a “through wafer via” 41. The cell 20 is kept in an appropriate electrophysiological buffer solution (not shown) which is contacted by the extra-cellular reference electrode 44 located on the first side of the substrate. The reference electrode could also be an external electrode which is brought in contact with the buffer solution prior to the measurement.

The intra- 40 and extra-cellular 44 electrodes are electrically isolated from each other, by the seal formed between the cell and the surface around or on the intra-cellular electrodes 40, making it possible to apply an electrical potential difference across the cell membrane while measuring the current response through the ion channels located in the cell membrane. The electrical seal resistance should be high enough that the major part of the current passes through the cell membrane and only a negligible amount through any possible parasitic leaks between the two electrodes. The cells are cultivated directly onto the substrates prior to the electrophysiological measurements. The cells uptake the intra-cellular electrodes during the cultivation by e.g. some form of endocytosis and form a high resistance seal around the electrodes or to the surrounding substrate surface, thereby effectively isolating the two electrodes from each other.

The surface material 46, intended to provide an easy seal with the membrane, includes materials such as, but not limited to: Quartz and other different glass types, Silicon, plastics, ceramics, oxides, elemental or compound semiconductors such as, Si, Ge, GaAs, GaP, InAs, InSb, SiC, or GaN.

The intra-cellular electrodes 40 are made of conducting or semiconducting elongated nanosize materials, having dimensions suitable for the penetration of a cell membrane. The resistance of a single electrode is in the low kΩ range as typically seen when measuring on “one dimensional (1D) electronic systems”. By using a set of 1D electrodes in parallel, the total resistance drops according, rendering a very low resistance connection to the interior of the cell. The total resistance of set of parallel electrodes is given by:

$$\frac{1}{{R}_{TOT}} = {\sum }_{i = 1}^{n} \frac{1}{{R}_{i}} ,$$

where R_{TOT} is the total resistance of all of the parallel electrodes and Rᵢ is the resistance of an individual nanosize electrode. As seen from the above formula, the total resistance of e.g. a set of ten individual electrodes each having a resistance of 100 kΩ amounts to one tenth of the individual resistance, in this case 10 kΩ.

The fabrication of conducting tracks and via's used for lead-in connections and the electrical isolation of these can be made in numerous way, by a person skilled in the art of semiconductor processing.

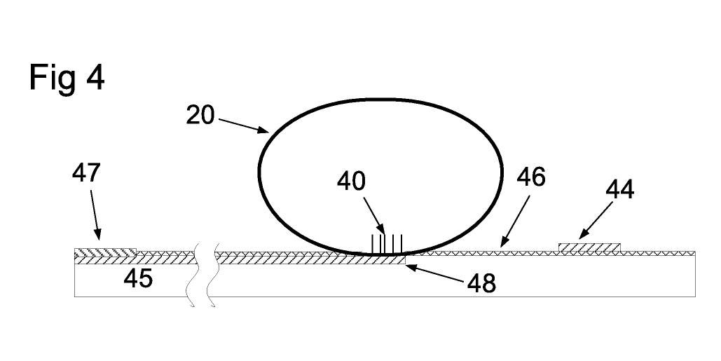

FIG. 4 basically shows the same design as in FIG. 3, but with the option of making the lead-in connection to the intra-cellular electrode 40 by a conducting track 48 on the first side of the substrate instead of using a via as in FIG. 3. The conducting track could for instance be made by ion-implantation into a doped Silicon substrate and the electrical isolation from the substrate realised by a pn-junction. The conducting track 48 is contacted by a suitable metallic contact 47. The extra cellular electrode 44 is located on the first side of the substrate.

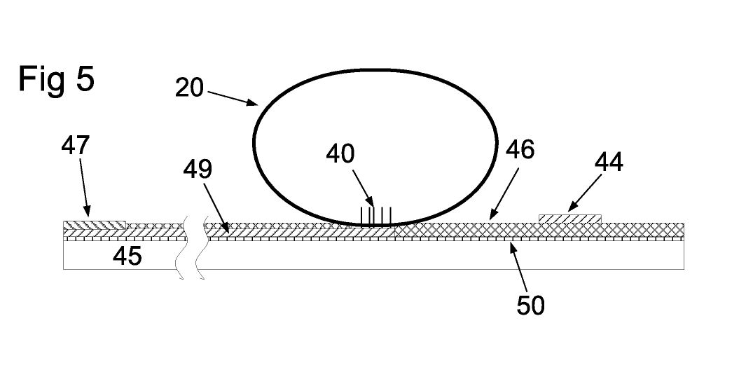

FIG. 5 is yet another revision of the design, wherein the conducting track 49 is isolated by the use of a suitable material 50 deposited onto or grown on the substrate. The isolator 50 could e.g. be a Silicon-dioxide layer and the conductor 49 could be a polysilicon layer or another suitable conductor. The structure thus resembles Silicon-On-Insulator (SOI) structures well know to people skilled in the art. The mentioned materials are only intended as examples; many other materials may be equally well suited.

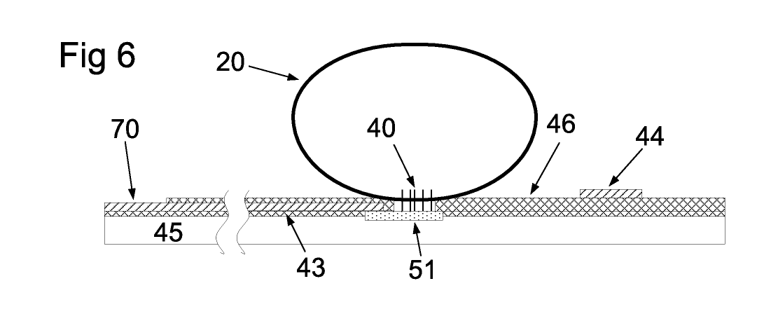

FIG. 6 shows a device where the intra-cellular electrodes 40 are grown on a p-type or n-type doped region 51 embedded in the semiconductor substrate material 45. The lead-in connector 70 contacts the doped region 51 thereby enabling a connection from external electronic circuits to the intra-cellular electrodes 40. Electrical isolation of the doped region 51 could e.g. be realised by a pn-junction. Isolation of the lead-in connector 70 could be made by deposition of suitable materials under 43 and over 46 the conductor. The isolator 46 could also serve as a suitable surface for the gigaseal formation.

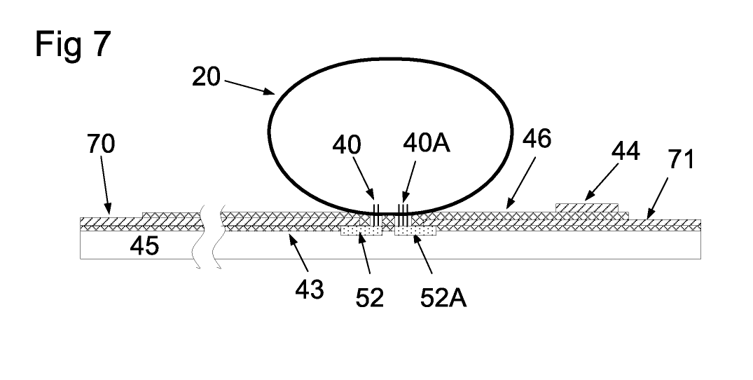

FIG. 7 is an example of a three point probe technique applied to a patch clamp measurement. In this configuration the cell is contacted by two sets of independent intracellular electrodes 40 and 40A. The two electrode sets are each contacted via separately doped regions 52 and 52A located in the substrate. These regions are again separately contacted by two lead-in conductors 70 and 71. The lead-in conductors are isolated by appropriate materials 43 and 46 encapsulating the conductors. The extra cellular electrode 44, located on the first side, could e.g. be a standard Ag/AgCl electrode.

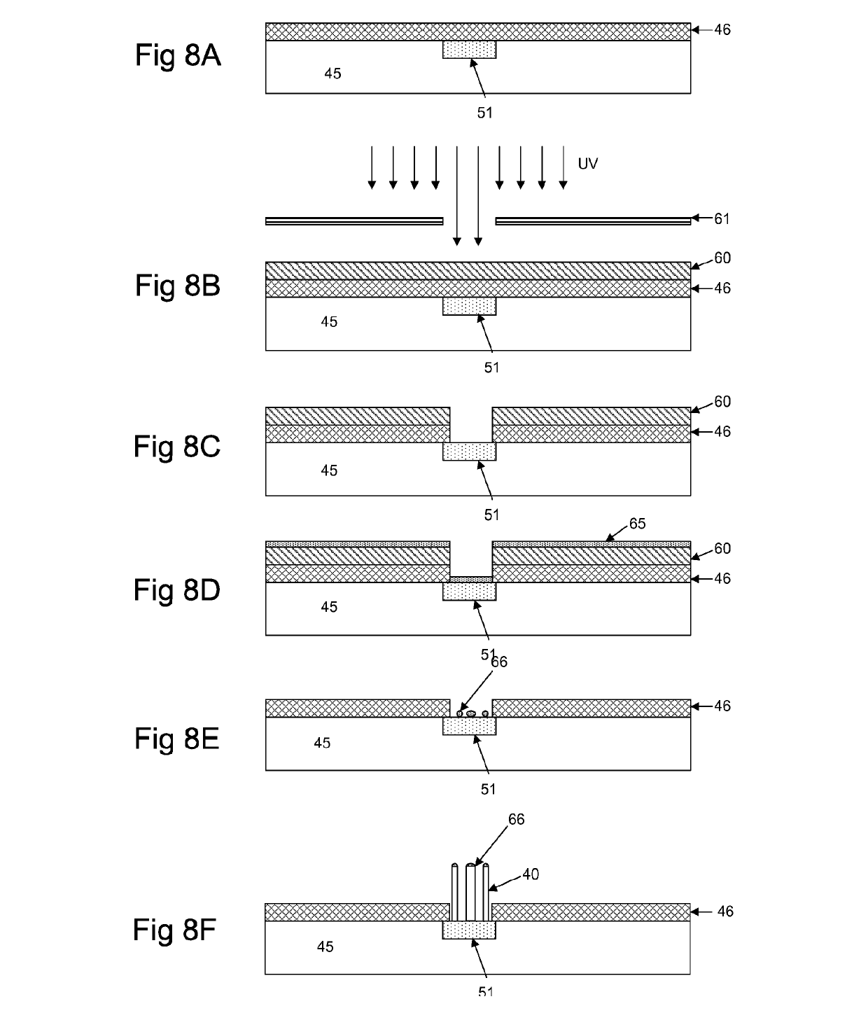

FIG. 8A-8F outlines a simple example of the main procedural steps required for positioning the elongated nanosized electrodes in specific locations on a substrate. Standard methods used in the art, such as photolithography, etching and deposition will be required for this example of one embodiment.

FIG. 8A shows a semiconductor substrate 45 with a predefined doped region 51 and covered with a suitable surface coating such as SiO₂ 46. The substrate is then covered with a photoresist 60 and exposed to UV-light through a mask 61 defining a selected area, as shown in FIG. 8B. After development and etching of the surface coating, the doped region 51 is exposed as shown in FIG. 8C. A subsequent cleaning step by e.g. an oxygen plasma, followed by a physical vapour deposition of a suitable amount of gold, leaves a substrate as shown in FIG. 8D, covered by a thin gold film 65, on top of the entire surface. By immersion in acetone, the photoresist and the gold on top of the resist can be removed by “lift-off”. The remaining gold is now located only in the openings to the doped regions 51.

FIG. 8E shows the substrate after alloying in a vacuum or inert gas. The heat treatment has made the gold located on the doped regions aggregate into small particles 66, with a diameter typically between 1 and 100 nm. The gold particles act as catalysts for the subsequent growth of the elongated nanosize electrodes. FIG. 8F shows the structure after the selective growth of the electrodes 40 in the predefined areas.

In one example the elongated nanosize electrodes are grown by Molecular Beam Epitaxy (MBE). The elongated nanosize electrodes are hereafter denoted: “Nano-wires” or “wires” for simplicity.

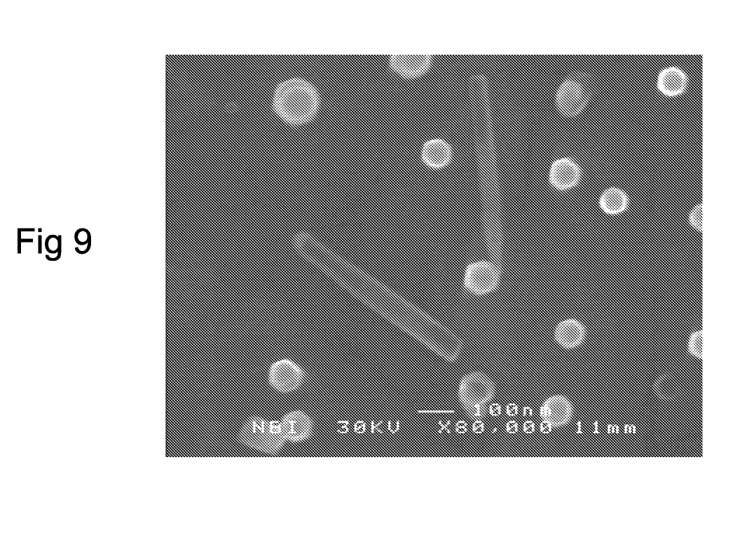

A solid source Varian GEN II system was used to realise the structures shown in FIG. 9. This figure shows a scanning electron micrograph of GaAs nanowires grown by MBE. The nanowires were grown on a GaAs substrate with a surface orientation of (111)B (the white scale bar is 100 nm). The SEM photo was taken along the (111) direction, the wires are therefore viewed along their longitudinal axis. The photo shows the often observed hexagonal cross-section of the wires with the gold catalyst particle on the top of the wires. Two broken off wires are shown lying on the substrate.

Prior to growth of the wires, the “epi-ready” substrate was loaded into the vacuum load-lock of the MBE system. After evacuation to a pressure below 5×10⁻⁷ torr, the substrate was degassed at 200° C. for a minimum of 1 hr. After degassing in the load-lock the pressure was allowed to recover to the low 10⁻⁷ torr range before transfer to the buffer chamber. In the buffer chamber the substrate was further degassed at 400° C. for 4 hrs. After cooling the substrate, the pressure dropped below 2×10⁻¹⁰ torr, and the substrate was transferred to the growth chamber.

The MBE growth chamber has a base pressure of 5×10⁻¹¹ torr. During growth, the substrate was exposed to a constant As₂ flux, when the substrate temperature was above 400° C. The Beam Equivalent Pressure of As₂ was about 1×10⁻⁵ torr. The substrate was heated to 660° C. in an As₂ flux to desorb the surface oxide. A gold film corresponding to about 1 nm thickness was deposited directly onto the warm surface at 660° C. and left to anneal for 10 min. The substrate temperature was then lowered to the growth temperature of about 510° C. GaAs was grown on the substrate with a rate of 0.6 μm/hr for about 20 min. The growth rate was calibrated prior to the nanowire growth on a separate GaAs (100) calibration piece. After growth, the sample was cooled under an As₂ flux. The length of the wires was 0.5-1 μm.

What is claimed is:

1. A biochip for measurement of electrophysiological properties of ion channels comprising:
a. a substrate having a first side and second side, where the surface of the first side is suitable for cultivation and adhesion of living cells.
b. one or a plurality of individual sites on the first side, each comprising a single or a group of elongated nanosize electrode structures suitable for the penetration of, or uptake by, the biological membranes, each electrode site and adjacent surface being capable of forming a high resistance seal with a biological membrane, thereby isolating the intra-cellular electrode structures penetrating the cell membrane from a reference electrode in contact with the extra-cellular saline solution.
c. one or a plurality of electrical connection leads connecting the elongated nanosize electrode structures to an appropriate electronic measurement setup, using standard processing techniques known in the art of thin film and semiconductor processing, such as photolithography, etching and suitable deposition techniques.
d. one or more reference electrodes located either on the first side of or outside the biochip, but in both cases in electrical contact with the carrier liquid.

2. The biochip of claim 1 further comprising a surface coating or chemical surface modification of all, or parts of the first surface, with the purpose of enhancing the adherence and growth of cells thereon.

3. A biochip according to any of the preceding claims further comprising a second surface coating or chemical surface modification of all, or parts of the first surface, with the purpose of enhancing the uptake of the elongated nanosize working electrodes.

4. A biochip according to any of the preceding claims wherein the substrate is made of an insulating material, semiconducting material or a combination thereof.

5. A biochip according to any of the preceding claims wherein the substrate is made of glass, plastic, ceramic, oxide or a combination thereof.

6. A biochip according to any of the preceding claims wherein the substrate is made of Si, Ge, GaAs, GaP, InAs, InSb, SiC, GaN or a combination thereof.

7. A biochip according to any of the preceding claims wherein the single or group of elongated nanosize electrode structures are made of one or a combination of conducting or semiconducting materials.

8. A biochip according to any of the preceding claims wherein the single or group of elongated nanosize electrode structures are made of one or a combination of conducting materials such as, elemental metals like Ag, Au, Pt, or Ni, or metallic alloys or metallic halides such as, AgCl.

9. A biochip according to any of the preceding claims wherein the single or group of elongated nanosize electrode structures are made of one or a combination of semiconducting materials such as, any elemental or compound semiconductor, for instance Si, Ge, GaAs, GaP, InAs, InSb, SiC, or GaN.

10. A biochip according to any of the preceding claims wherein the single or group of elongated nanosize semiconducting electrode structures are doped either n-type or p-type or a combination thereof.

11. A biochip according to any of the preceding claims wherein the single or group of elongated nanosize electrode structures are protruding from the substrate at angles between 0.1° and 90°.

12. A biochip according to any of the preceding claims wherein the single or group of elongated nanosize electrode structures are characterized by an outer diameter of 0.8 nm to 1000 nm and a length of 10 nm to 10 μm.

13. A biochip according to any of the preceding claims wherein the single or group of elongated nanosize electrode structures are characterized by an outer diameter of 20 nm to 200 nm and a length of 0.2 μm to 4 μm.

14. A biochip according to any of the preceding claims wherein the single or group of elongated nanosize electrode structures are coated with a metal such as but not limited to Ag.

15. A biochip according to any of the preceding claims wherein the single or group of elongated nanosize electrode structures are characterized by having an average spacing between two independent electrode sites larger than a typical cell diameter of the cells used.

16. A biochip according to any of the preceding claims wherein the electrode structures are characterized by having pairs of electrode sites spaced by a distance less than a typical cell diameter, and where each said pair of electrode sites are spaced by more than a typical cell diameter from neighbouring pairs of electrode sites.

17. A biochip according to any of the preceding claims wherein said single or group of elongated nanosize electrode structures are made from conducting or semiconducting single- or multi-wall carbon nanotubes or nanorods/whiskers, by methods commonly used by persons skilled in the art.

18. A biochip according to any of the preceding claims wherein said single or group of elongated nanosize electrode structures are made from conducting or semiconducting nanorods/whiskers, by methods such as, arc-evaporation, chemical vapour deposition, molecular beam epitaxy, chemical beam epitaxy, liquid phase epitaxy or by a combination of said methods.
